(19) United States
(12) Patent Application Publication
(10) Pub. No.: US 2018/0218779 A1
(43) Pub. Date: Aug. 2, 2018

(54) VERIFICATION OF CLINICAL DATA

(71) Applicant: OpenClinica LLC, Waltham, MA (US)

(72) Inventor: Michael T. Collins, JR., Needham, MA (US)

(21) Appl. No.: 15/876,381

(22) Filed: Jan. 22, 2018

Related U.S. Application Data
(60) Provisional application No. 62/453,156, filed on Feb. 1, 2017.

(51) Int. Cl. G16H 10/20; G06Q 20/38; H04L 9/06; H04L 9/32

(52) U.S. Cl. CPC G16H 10/20; G06Q 20/3827; H04L 9/0643; G06Q 20/0655; H04L 9/3247; H04L 9/3297; G06Q 2220/10; H04L 9/3236

(57) ABSTRACT

A method for securely storing clinical data associated with a clinical trial in a data store includes receiving the clinical data from a user, forming a value representing the clinical data including applying a one-way function to the clinical data, storing the value in a distributed ledger, and storing the clinical data in the data store.

### CROSS REFERENCE TO RELATED APPLICATIONS

This application claims the benefit of Provisional Application Ser. No. 62/453,156, filed Feb. 1, 2017, the contents of which are hereby entirely incorporated herein by reference.

### BACKGROUND

This invention relates to secure maintenance and verification of clinical research data.

In general, clinical trials are experimental studies that are designed to answer specific questions about new treatments such as novel vaccines and drugs or known treatments that warrant further study. Among other goals, clinical trials are conducted to generate data related to the safety and efficacy of the treatments. Ultimately, the parameters and outcomes of clinical trials are analyzed by an authority such as the U.S. Food and Drug Administration (FDA) to determine whether treatments are appropriate for wide scale use in humans, require further study before wide scale use in humans, or are unsafe for wide scale use in humans.

Clinical trials are typically initiated by a sponsor (e.g., a pharmaceutical company) which has developed a new treatment that they would like to sell. In general, a clinical site provides the collected clinical research data to the sponsor, but is required by the FDA to maintain independent copies of the clinical research data. In this way, the FDA's clinical trial monitors can verify that the sponsor has not tampered with or altered the clinical research data to influence the outcome of the clinical trial.

### SUMMARY

In a general aspect, a method for securely storing clinical data associated with a clinical trial in a data store includes receiving the clinical data from a user, forming a value representing the clinical data including applying a one-way function to the clinical data, storing the value in a distributed ledger (for example, a blockchain based distributed ledger or, more specifically, the bitcoin blockchain based distributed ledger), and storing the clinical data in the data store.

Aspects may include one or more of the following features.

Storing the value in the blockchain based distributed ledger may include providing the value to a computing node of a plurality of computing nodes associated with the blockchain based distributed ledger, wherein the computer forms a block for addition to the blockchain distributed ledger, the block including a representation of the value. The method may include receiving a confirmation that the block including the representation of the value has been permanently added to the blockchain based distributed ledger.

The value may further represent a first digital signature associated with the user and forming the value may include applying the one-way function to a combination of the clinical data and the first digital signature. The value may further represent a second digital signature associated with computer code used to form the value and forming the value may include applying the one-way function to a combination of the clinical data, the first digital signature, and the second digital signature. The method may include receiving the computer code used to form the value from a trusted third party. The one-way function may be a cryptographic hash. The data store may be under the control of a sponsor of the clinical trial. Storing the value in the distributed ledger may include forming a transaction for transferring a quantity of currency to an address corresponding to the value. The distributed ledger may include the Bitcoin blockchain based distributed ledger.

In another general aspect, a method for verifying a clinical data record associated with a clinical trial and stored in a data store includes receiving the clinical data record from the data store, forming a first value representing the received clinical data record including applying a one-way function to the received clinical data record, receiving a transaction record associated with the clinical data record from a distributed ledger, the transaction including a second value, and comparing a second value included in the transaction record to the first value to determine whether the first value and the second value match.

Aspects may include one or more of the following features.

The method may include receiving a first digital signature associated with a clinical user from the data store, wherein the first value further represents the first digital signature and forming the first value includes applying the one-way function to the first digital signature. The method may include receiving a second digital signature associated with computer code used to form the second value, wherein the first value further represents the second digital signature and forming the first value includes applying the one-way function to the second digital signature. The one-way function may include a series of one or more a cryptographic hash functions. The data store may be under the control of a sponsor of the clinical trial. The distributed ledger may include the Bitcoin blockchain based distributed ledger.

In another general aspect, a computing system for securely storing clinical data associated with a clinical trial in a data store includes a data store for storing clinical data, an input port configured to receive the clinical data from a user, at least one processor configured to form a value representing the clinical data including applying a one-way function to the clinical data, store the value in a distributed ledger, and store the clinical data in the data store.

In another general aspect, software for securely storing clinical data associated with a clinical trial in a data store is stored in a non-transitory form on a computer-readable medium and includes instructions for causing a computing system to receive the clinical data from a user, form a value representing the clinical data including applying a one-way function to the clinical data, store the value in a distributed ledger, and store the clinical data in the data store.

One common way of collecting clinical research data at a clinical site is to use electronic data capture (EDC) techniques. EDC techniques typically collect clinical research data using web-based software executing on web servers. A key shortcoming of EDC techniques is that clinical research data is typically submitted to a sponsor-controlled system without first being independently recorded and stored at the clinical site, which is non-compliant with the FDA's clinical trial regulations. For example, some web based EDC techniques receive clinical resource data in an electronic case report form (eCRF) and then transmit the clinical resource data directly to a sponsor (or a sponsor's delegated vendor) controlled central server. Even if a clinical investigator at the clinical site immediately receives a copy of the clinical research data from the sponsor-controlled central server, it has already been under the sponsor's control and therefore been subject to tampering or alteration.

Electronic patient reported outcome (ePRO) and electronic clinical outcome assessment (eCOA) technologies operate under a similar paradigm as eCRF technologies and therefore suffer from the same regulatory non-compliance issues. These techniques may save clinical research data to a mobile device prior to transmission to the sponsor-controlled central server. However, they more typically submit clinical research data directly to the sponsor-controlled central server and, even if the clinical research data is saved at a mobile device, doing so as a basis for long term archiving of clinical research data by clinical sites is logistically difficult and technically unreliable.

As a result of the regulatory compliance issues associated with eCRF, ePRO, and eCOA techniques, many clinical sites have reverted to maintaining redundant paper copies of the records or to using a conventional client-server data capture model in which the client site collects and maintains data locally. Reverting to conventional techniques is seen as a step backwards in efficiency by many in the clinical research field and is also not a foolproof way of ensuring compliance.

In the United States, the above-described regulatory issues are primarily directed to the maintenance of electronic source (eSource) clinical research data. However, in many countries around the world the issues extend to transcribed case report form (CRF) data. For example, European regulators note that “[i]t is essential that the principal investigator (PI) maintains an independent (i.e. out of the sponsor's control), contemporaneous copy of the CRF for which he has exclusive control. This could be at the PI site, although, it could also be maintained at an independent site/vendor when assuring the investigator's continuous access” (see http://www.ema.europa.eu/docs/en_GB/document library/Minutes/2014/01/WC500159409.pdf).

That is, the responsibility for archiving and maintaining the CRF at the investigator's clinical site, regardless of whether it is in paper or electronic form, is the responsibility of the investigator and not that of the sponsor.

The European Medical Agency's Good Clinical Practice (GCP) Inspector's Working Group (IWG) has noted that the current state of electronic data capture systems does not fulfill current regulatory requirements and is therefore unacceptable for use in collection of clinical research data. The IWG acknowledged that solutions may take time to develop and indicated that, in the meantime sponsors should implement mitigating actions since any solution where the server is under the control of the sponsor is not acceptable and has been implemented without ensuring that the systems are in line with current regulatory requirements.

The above-described regulatory problems arose from the realm of computer networks, because it is computer networks (e.g., the internet) that, at least in part, made eCRF, ePRO, and eCOA techniques possible. So, aspects described herein are necessarily rooted in computer technology in order to overcome the above-described regulatory problems.

Aspects described herein implement techniques for electronically capturing clinical research data in a secure manner by timestamping the clinical research data and using a distributed ledger, such as the Bitcoin network, to record the timestamps. In particular, aspects retain the original record of clinical research data (or a certified copy) under the control of the investigator at the clinical site and are also able to verify who created the record (i.e., the investigator, a CRC, a study participant) using the distributed ledger. In doing so, aspects directed to providing control over the security and validity of individual pieces of information released to sponsors over a network and facilitate the use of electronic clinical records in a networked environment. That is, aspects are directed to the use of blockchain based distributed ledgers in networked environment and solve the problem of information security and validity (as it pertains to electronic clinical records) by including a mechanism to leverage blockchain based distributed ledgers to ensure information security and validity.

In some aspects, a decentralized (e.g., blockchain based) trusted timestamping technique is used to verify that a copy of a clinical research data record (stored on an electronic data capture server, or anywhere else) is exactly the same as the original clinical research data record in the investigator's web browser, even if the original record no longer exists. In particular, trusted timestamping is used to prove that the investigator (or designee, such as a nurse or clinical research coordinator), at the clinical site possessed a document, some information, or a file at a specific point in time, in a way that can't be forged.

Many conventional trusted timestamping techniques require trusted timestamps that are issued by trusted third parties called Time Stamping Authorities (TSAs). TSAs are notoriously prone to data corruption and tampering. The decentralized (e.g., blockchain based) trusted timestamping techniques used by aspects described herein obviate the need for a central trusted party. That is, a blockchain based timestamp is safely stored in a distributed manner in a public ledger, and is much more difficult to corrupt or tamper with.

In addition to being able to verify that a copy of a clinical research data record is exactly the same as the original clinical research data record, aspects described herein are also able to verify that it was the investigator (or patient) that created the clinical research data record. To do so, a trusted digital signature associated investigator (or patient) is also embedded in the trusted timestamping technique. With the investigator's (or patient's) digital signature embedded in the trusted timestamping technique, a certified copy of the original clinical research data record can be retrieved or regenerated at any time. As long as the certified copy matches, byte-for-byte, the original clinical research data record, the copy can be verified as matching the original clinical research data record as it was submitted by the investigator at the client site.

Aspects may include one or more of the following advantages:

Aspects are able to verify the origin and contents of clinical research data without the need to rely on time stamping authorities. Aspects allow clinical sites to rely on the authenticity and integrity of the clinical research data without requiring that expensive, complex archiving software is installed on-site.

Other features and advantages of the invention are apparent from the following description, and from the claims.

### DESCRIPTION OF DRAWINGS

FIG. 1 is a system for collecting clinical research data.

FIG. 2 is a detailed schematic diagram of the client site of FIG. 1.

FIG. 3 is a detailed schematic diagram of the sponsor site of FIG. 1.

### DESCRIPTION

### 1 System Overview

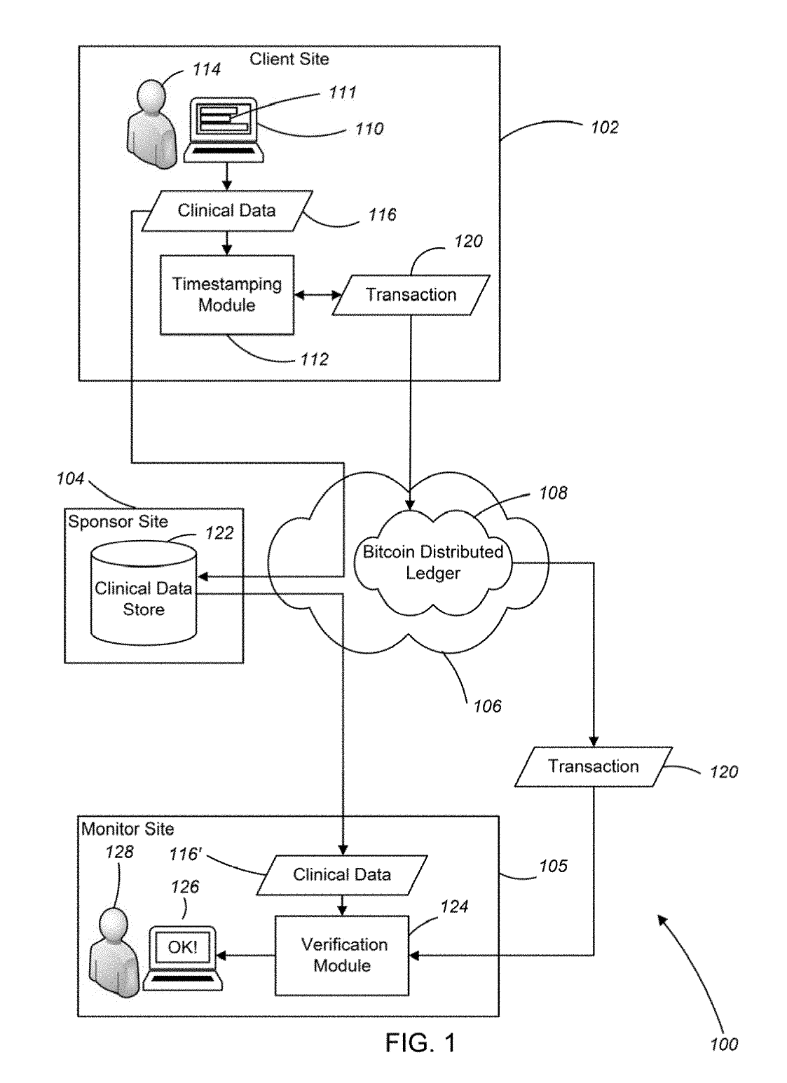

Referring to FIG. 1, a system for collecting clinical research data 100 includes a client site 102, a sponsor site 104, and a monitor site 105. The client site 102, the sponsor site 104, and the monitor site 105 are in communication with each other over a communications network 106 (e.g., over the Internet). Both the client site 102 and the monitor site 104 are also in communication with the Bitcoin blockchain based distributed ledger 108. Very generally, the system 100 collects and stores clinical research data such a way that the content and originator of the clinical research data can be verified and any modification of the clinical research data that occurs after its time of collection can be detected.

The client site 102 (e.g., a medical office) includes an input device 110 (e.g., a personal computer or a mobile device) and a timestamping module 112. As is described in greater detail below, a clinical research data input form 111 is presented to a user 114 (e.g., a clinical investigator or a patient) via the input device 110. The user 114 completes the clinical research data input form 111 using the input device 110 and the completed form is used to generate an original clinical research data record 116. The timestamping module 112 processes information including the original clinical research data record 116 to form a transaction address 120 including a Bitcoin address that is computed as a cryptographic hash generated from the contents of the original clinical research data record 116, a digital signature of the code used to present the input form 111, and a digital signature of the creator of the record. The hash is then submitted to and stored in the Bitcoin blockchain based distributed ledger 108 by using the hash as the address for a Bitcoin transaction.

The original clinical research data record 116 (among other information, as is described below) is transmitted over the communication link 106 to the sponsor site 104 (e.g., a pharmaceutical company site) where it is stored as a stored clinical research data record 116′ in a clinical data store 122.

The monitor site 105 includes a verification module 124 and a monitor device 126 (e.g., a personal computer or a mobile device). A monitor user 128 (e.g., a regulatory official from the FDA) accesses the monitor device 126 to verify that the clinical research data record stored at the sponsor site 104 has not been altered and to verify the origin of the clinical research data record. To perform this verification, a stored clinical research data record 116′ (including its timestamp and digital signatures) is requested from the clinical data store 122 at the sponsor site 104 and the transaction address 120 including the timestamp associated with the original clinical research data record 116 is read from the Bitcoin blockchain based distributed ledger 108.

The stored clinical research data record 116′ and the transaction address 120 are provided to the verification module 124 which computes a cryptographic hash from the stored clinical research data record 116′ and compares the computed hash to the Bitcoin address of the transaction 120. If the two hashes match, then the verification module 124 indicates that the stored clinical research data record 116′ matches the original clinical research data record 116 (i.e., no alteration of the clinical research data record has occurred). If the clinical research data record 116′ has been altered by even one byte the hashes will not match. Otherwise, if the two hashes differ, then the verification module 124 indicates (via the monitor device 126) that the stored clinical research data record 116′ does not match the original clinical research data record 116 (i.e., alteration of the clinical research data record has occurred).

The monitor user 128 uses the monitor device 126 to verify some or all of the clinical research data records stored in the clinical research data store 122 and assesses the validity of the clinical trial based on the verification results.

### 2 Client Site

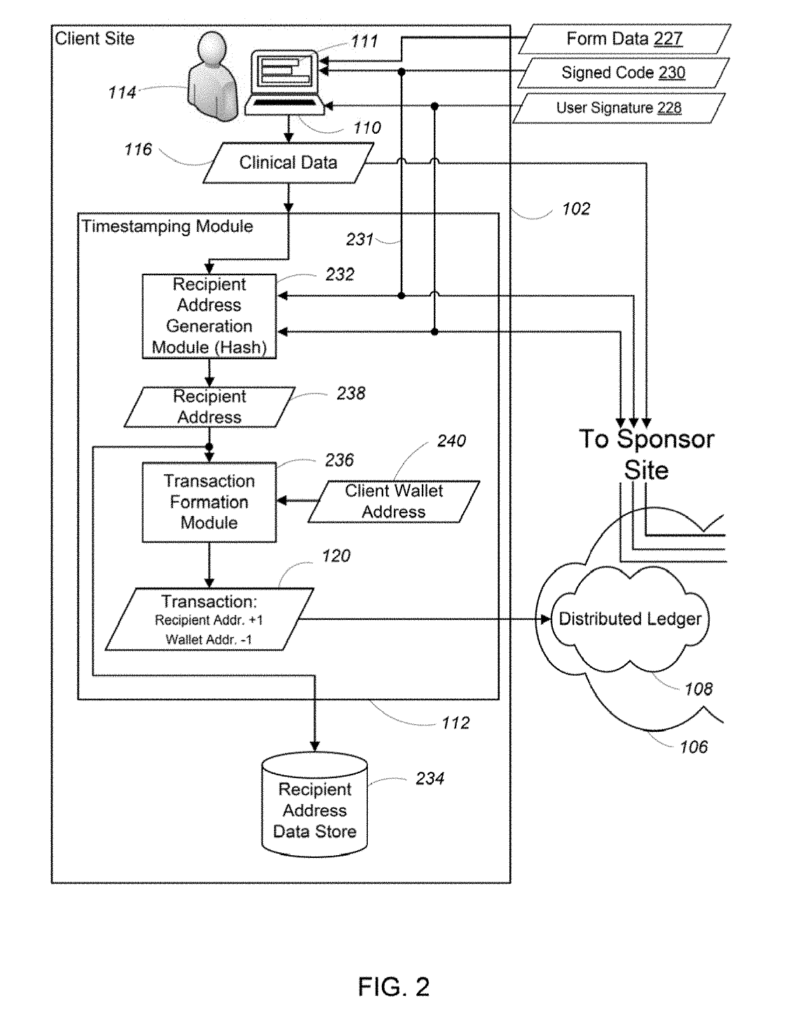

Referring to FIG. 2, a detailed schematic diagram of the client site 102 includes the user device 110, the timestamping module 112, and a recipient address data store 234. The client site 102 enters form data 227 for presentation to the user 114 via the clinical research data input form 111, a user digital signature 228 associated with the user 114, and signed code 230 with a strict content security policy for implementing the timestamping module 112. The client site 102 uses the form data 227, the user digital signature 228, and the signed code 230 to generate the original clinical research data record 116 from the user 114 in a secure manner and to form the transaction 120. In some examples, the clinical research data input form 111 is securely received from the provider site 104, the user digital signature 228 is received from an identity provider, and the signed code 230 is received from a trusted third party such as github.

When collection of the original clinical research data record 116 and formation of the transaction 120 is complete, the client site 102 provides the original clinical research data record 116, an original code digital signature 231 associated with the signed code 230, and the original user digital signature 228 to the sponsor site 104 for storage. The sponsor site 104 stores the provided information in the clinical data store 122 as a stored clinical research data record 116′, a stored code digital signature 231′, and a stored user digital signature 228. The client site 102 also submits the transaction 120 to the Bitcoin blockchain based distributed ledger 108.

### 2.1 Client Site Configuration

Once received, the form data 227 is used by the input device 110 to present the clinical research data input form 111 to the user 114.

The signed code 230 is also received by the input device 110, which processes the signed code 230 to implement the timestamping module 112 (e.g., as a subroutine executing on the user device 110). The timestamping module 112, as configured according to the signed code 230, includes a recipient address generation module 232 and a transaction formulation module 236. In some examples, the processing of the signed code 230 includes verifying a code digital signature associated with the signed code 230 to ensure that the signed code 230 is authentic.

### 2.2 Client Site Operation

In operation, when the user 114 completes the clinical research data input form 111 at the user device 110, the user device 110 generates the original clinical research data record 116 based on the information provided by the user 114 in the form 111. The code digital signature 231 associated with the signed code 230, the user digital signature 228, and the original clinical research data record 116 are provided as input to the timestamping module 112.

In the timestamping module 112, the recipient address generation module 232 receives the original clinical research data record 116, the signature associated with the signed code 230, and the user digital signature 228 as input and processes the received input using a one-way function (e.g., a cryptographic hash function) to generate a recipient address 238.

The recipient address 238 and a sender wallet address 240 associated with the client site 102 are provided to the transaction formation module 236 which processes the two addresses 238, 240 to form the Bitcoin transaction 120. A timestamp for the transaction is also included by the transaction formation module 236. In some examples, the transaction 120 includes a transfer of 1 Satoshi (i.e., the smallest denomination of a Bitcoin available on the Bitcoin network) from the Bitcoin wallet associated with the client wallet address 240 to the Bitcoin wallet associated with the recipient address 238. That is, the purpose of the transaction is not to establish a substantial transfer of funds, but is instead to add a small transaction to the Bitcoin blockchain, the small transaction including the recipient address 238 which can be used at a later date to verify the validity of a stored version of clinical research data record 116.

The transaction 120 is added to the Bitcoin blockchain based distributed ledger 108 according to the well known and established Bitcoin transaction protocol. The transaction formation module 236 periodically queries the Bitcoin network until the transaction is confirmed.

Finally, in some examples, some or all of the recipient address 238, the transaction identifier and the transaction block hash returned from the Bitcoin network, and/or the Bitcoin transaction itself are stored in the recipient address data store 234. The recipient address stored in the recipient address data store 234 can later be recalled to quickly verify clinical research data.

### 3 Monitor Site

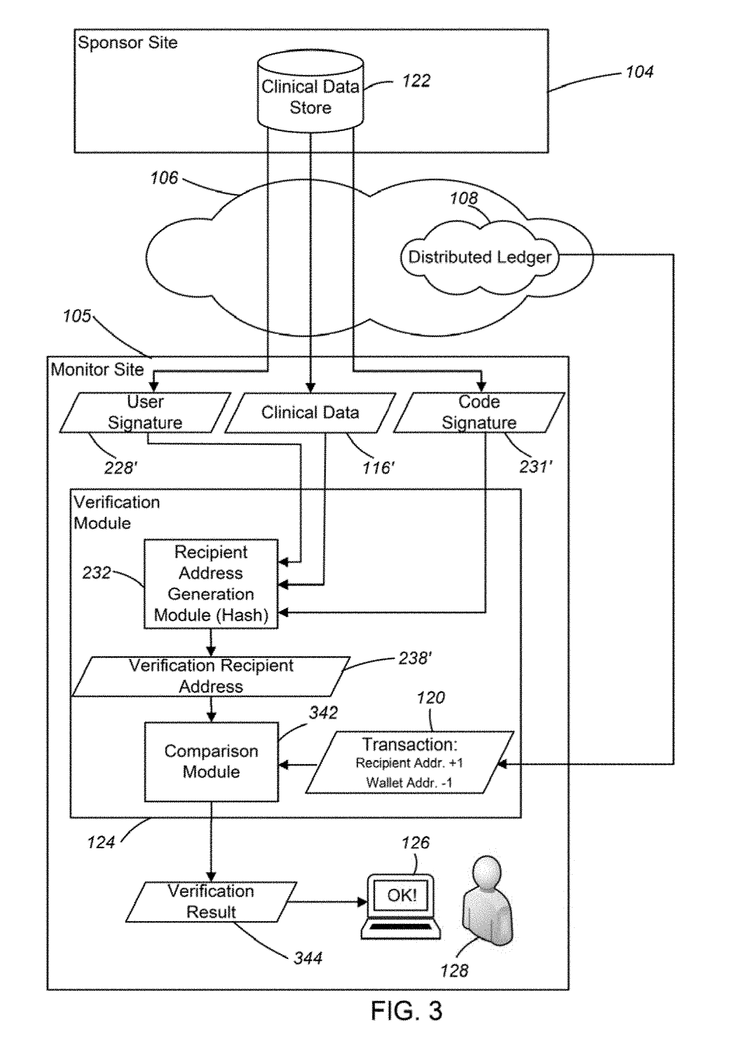

Referring to FIG. 3, a detailed schematic diagram of the monitor site 105 includes the monitor device 126 and the verification module 124. The monitor site 105 receives as input the stored clinical research data record 116′, the stored code digital signature 231′ associated with the signed code 230, and the stored user digital signature 228′ from the sponsor's clinical data store 122. The monitor site 105 also receives the transaction 120 associated with the original clinical research data record 116 from the Bitcoin blockchain based distributed ledger 108. The monitor site 105 processes its inputs to verify the validity and origin of the stored clinical research data record 116′.

### 2.1 Monitor Site Operation

When the monitor user 128 requests verification of the stored clinical research data record 116′ from the sponsor site 104 using the monitor device 126, the stored clinical research data record 116′, the stored code digital signature 231′, and the stored user digital signature 228′ are retrieved from the sponsor site's clinical data store 122. The monitor device 126 also requests the transaction 120 associated with the original clinical research data record 116 from the Bitcoin blockchain based distributed ledger 108.

Once received at the monitor site 105, the stored clinical research data record 116′, stored code digital signature 231′, and the stored user digital signature 228′ are provided to the verification module 124. The verification module 124 includes the recipient address generation module 232 and a comparison module 342.

The stored clinical research data record 116′, the stored code digital signature 231′, and the stored user digital signature 228′ are provided as input to the recipient address generation module 232. The recipient address generation module 232 processes the received input using a one-way function (e.g., a cryptographic hash function) to generate a verification recipient address 238′.

The verification recipient address 238′ is provided to the comparison module 342 along with the transaction 120 associated with the stored clinical research data record 116′. The comparison module 342 compares the original recipient address 238 stored in the transaction 120 with the verification recipient address 238′ to determine whether they match. If the two recipient addresses match, then the comparison module 342 generates a verification result 344 indicating that no alteration of the stored clinical research data record 116′, the stored code digital signature 231′, or the stored user digital signature 228′ has occurred. Otherwise, if the two recipient addresses do not match, then the comparison module 342 generates a verification result 344 indicating that one or more of the stored clinical research data record 116′, the stored code digital signature 231′, or the stored user digital signature 228′ has been altered.

The verification result 344 is provided to the monitor device 126 where it is displayed to the monitor user 128 for evaluation.

### 4 Alternatives

In some examples, the clinical site is located at a hospital or medical clinic. In other examples, the clinical site is a location of a patient when they enter their clinical data using, for example, a mobile device or personal computer.

In some examples, the sponsor site is located on the premises of a pharmaceutical company's offices. In some examples, the sponsor site is located in the cloud (e.g., the Amazon cloud). In some examples, the sponsor site is maintained by a third party on behalf of the sponsor.

In some examples, the monitor site is located at a regulatory agency (e.g., the FDA) that is responsible for monitoring clinical trials. In other examples, the monitor site can be co-located with either the sponsor site or the client site. In other examples, the monitor site is located wherever the monitor user has access to the sponsor site and the distributed ledger.

While the above embodiment is described in the context of using the Bitcoin block chain, other distributed ledgers/block chains (e.g., Etherium, Kadena, Bitcoin Cash, Ripple, and Litecoin) can also be used to implement the system. For example, privately owned and maintained blockchain based distributed ledgers can be used for a fee.

In some examples, some or all of the communication that takes place over the communication network is secure (e.g., uses the HTTPS protocol).

In some examples, the recipient address data store is located at the sponsor site rather than at the client site, as is described above.

It is to be understood that the foregoing description is intended to illustrate and not to limit the scope of the invention, which is defined by the scope of the appended claims. Other embodiments are within the scope of the following claims.

What is claimed is:

1. A method for securely storing clinical data associated with a clinical trial in a data store, the method comprising:
receiving the clinical data from a user;
forming a value representing the clinical data including applying a one-way function to the clinical data;
storing the value in a blockchain based distributed ledger; and
storing the clinical data in the data store.

2. The method of claim 1 wherein storing the value in the blockchain based distributed ledger includes providing the value to a computing node of a plurality of computing nodes associated with the blockchain based distributed ledger, wherein the computer forms a block for addition to the blockchain distributed ledger, the block including a representation of the value.

3. The method of claim 2 further comprising receiving a confirmation that the block including the representation of the value has been permanently added to the blockchain based distributed ledger.

4. The method of claim 1 wherein the value further represents a first digital signature associated with the user and forming the value includes applying the one-way function to a combination of the clinical data and the first digital signature.

5. The method of claim 1 wherein the value further represents a second digital signature associated with computer code used to form the value and forming the value include applying the one-way function to a combination of the clinical data, the first digital signature, and the second digital signature.

6. The method of claim 5 further comprising receiving the computer code used to form the value from a trusted third party

7. The method of claim 1 wherein the one-way function is a cryptographic hash.

8. The method of claim 1 wherein the data store is under the control of a sponsor of the clinical trial.

9. The method of claim 1 wherein storing the value in the blockchain based distributed ledger includes forming a transaction for transferring a quantity of currency to an address corresponding to the value.

10. The method of claim 1 wherein the blockchain based distributed ledger includes the Bitcoin blockchain based distributed ledger.

11. A method for verifying a clinical data record associated with a clinical trial and stored in a data store, the method comprising:
receiving the clinical data record from the data store;
forming a first value representing the received clinical data record including applying a one-way function to the received clinical data record;
receiving a transaction record associated with the clinical data record from a blockchain based distributed ledger, the transaction including a second value; and
comparing a second value included in the transaction record to the first value to determine whether the first value and the second value match.

12. The method of claim 11 further comprising receiving a first digital signature associated with a clinical user from the data store, wherein the first value further represents the first digital signature and forming the first value includes applying the one-way function to the first digital signature.

13. The method of claim 11 further comprising receiving a second digital signature associated with computer code used to form the second value, wherein the first value further represents the second digital signature and forming the first value includes applying the one-way function to the second digital signature.

14. The method of claim 11 wherein the one-way function includes a series of one or more a cryptographic hash functions.

15. The method of claim 11 wherein the data store is under the control of a sponsor of the clinical trial.

16. The method of claim 11 wherein the blockchain based distributed ledger includes the Bitcoin blockchain based distributed ledger.

17. A computing system for securely storing clinical data associated with a clinical trial in a data store, the computing system comprising:
a data store for storing clinical data;
an input port configured to receive the clinical data from a user;
at least one processor configured to
form a value representing the clinical data including applying a one-way function to the clinical data;
store the value in a blockchain based distributed ledger; and
store the clinical data in the data store.

18. Software for securely storing clinical data associated with a clinical trial in a data store, the software stored in a non-transitory form on a computer-readable medium and including instructions for causing a computing system to:
receive the clinical data from a user;
form a value representing the clinical data including applying a one-way function to the clinical data;
store the value in a blockchain based distributed ledger; and
store the clinical data in the data store.
